(19) United States
(12) Patent Application Publication
(10) Pub. No.: US 2008/0208240 A1
(43) Pub. Date: Aug. 28, 2008

(54) Implantable Device For Obesity Prevention

(75) Inventor: Adrian Paz, Petah Tikva (IL)

Correspondence Address: DARBY & DARBY P.C., P.O. BOX 770, Church Street Station, New York, NY, 10008-0770, US

(73) Assignee: STIMPLANT LTD., Kiryat Shmona (IL)

(21) Appl. No.: 12/063,134

(22) PCT Filed: Aug. 13, 2006
(86) PCT No.: PCT/IL06/00934

§ 371 Date: May 5, 2008

Related U.S. Application Data
(60) Provisional application No. 60/707,227, filed on Aug. 11, 2005.

(51) Int. Cl. A61M 29/02

(52) U.S. Cl. 606/191

(57) ABSTRACT

A device for controlling the expansion of a hollow internal organ, comprising an inflatable balloon, which is inserted in an uninflated state into the desired position close to the organ using minimally invasive procedures. After insertion, the balloon is inflated to its required size and shape. The invention is useful for restricting the expansion of the stomach during meals, thus inducing a feeling of satiety and preventing over-eating. Inflation may be performed through a catheter connected to a readily accessible inflation port. In the case of the gastric embodiment, the balloon may be positioned pro-peritoneally, such that the procedure is surgically simple. One or more sensors located close to the organ to be controlled, may monitor a physiological effect relating to the organ, and the output of the sensor used to control the level of inflation of the balloon in order to correct the condition being monitored.

### FIELD OF THE INVENTION

The present invention relates to the field of the treatment of obesity, especially by the use of inflatable devices implanted in the region of the stomach.

### BACKGROUND OF TH INVENTION

Obesity represents a significant burden on society. In the USA, it is estimated that approximately $100 Billion are spent each year in direct costs for the treatment of obesity and in indirect costs for the significant side effects of obesity on the cardiovascular system, skeletal system, and other anatomical systems, and the resulting hospitalizations, treatments and loss of working days.

The current most common surgical treatment for morbid obesity is based on constricting devices that are placed around the proximal part of the stomach in order to restrict the quantity of food ingested during each meal and to achieve a sensation of satiety. Such a device, known commercially as the Lap-Band® was first described in the article by Dr. Solhaug, entitled “Gastric Banding, A New Method in the Treatment of Morbid Obesity” published in Current Surgery, pp. 424-428, November-December, 1983, and aspects thereof were shown thereafter in U.S. Pat. No. 4,696,288.

Other modalities include various kinds of gastric bypass operations, in which the proximal end of the stomach is resected creating a small pouch. The addendum is again resected about 1 ft below the stomach. The distal part of this resection is anastomized to the above pouch and the proximal part is anastomized to the small intestine about 3-5 ft below the stomach. A further procedure is sleeve gastrectomy in which the whole stomach is resected by stapling along the small curvature, creating a reduced volume sleeve.

There is a trend to perform those of the above procedures where it is feasible using laparoscopic techniques, which incurs faster convalescence. Despite the advancements in laparoscopy, these operations are demanding, especially in the morbidly obese patients and are accompanied by a significant percent of side effects and complications, due to the surgery itself, and/or to the general anesthesia which has significant risks for these patients.

There therefore exists a need for an improved device and method for the prevention of obesity, which overcomes at least some of the disadvantages of the prior art methods and devices.

The disclosures of each of the publications mentioned in this section and in other sections of the specification, are hereby incorporated by reference, each in its entirety.

### SUMMARY OF THE INVENTION

The present invention seeks to provide a new device for controlling the expansion of a hollow internal organ. The device comprises an inflatable balloon, which is inserted in an uninflated state into the desired position by means of minimally invasive procedures using an introducing tube. After insertion, the balloon is inflated to its required size and shape through a catheter. The invention is particularly useful for restricting the expansion of the stomach during meals, thus inducing a feeling of satiety and preventing over-eating. The inflation is preferably performed through a catheter connected to a readily accessible inflation port. The port preferably consists of a self sealing chamber or reservoir that can be releasably attached to the catheter.

The balloon and catheter are preferentially made of a distendable material such as medical grade silicone, though other plastic materials may be used. The balloon preferentially inflates to a predetermined shape and size, selected to apply pressure preferably to a large portion of the wall of the organ in proximity to which it is placed, and operates as an anatomical implant. The balloon configuration is chosen to fit snugly into the anatomical space into which it is inserted, thus preventing excessive pressure on particular parts of the surrounding tissue, which could lead to ischemia and erosion and fistula formation. Additionally, the intended position should be such that the surrounding anatomy does not enable the balloon to move easily from its predetermined position. In the case of the gastric embodiment, the balloon, according to one preferred embodiment, is positioned pro-peritonealty, such that its motion is limited. In this case, the insertion procedure is surgically simpler than procedures in which the peritoneum is penetrated. According to another preferred embodiment, the gastric balloon is located behind the stomach, either in or close to the Morrison pouch, such that it cannot readily move out of position.

The catheter and balloon are preferably introduced into the desired position using a specially designed introducer kit, preferably comprising a needle, a guide wire, and a dilator. The deflated balloon and catheter are preferably enveloped by a sheath.

According to further preferred embodiments of the present invention, one or more sensors are located in proximity to the organ to be controlled by the device, the sensor or sensors monitoring a physiological effect relating to the organ to be controlled, the output of the sensor or sensors being used to control the level of inflation of the balloons in order to control the expansion of the organ being monitored or to correct a condition thereof.

According to another preferred embodiment of the present invention, the balloon is positioned next to the esophagus, such that when inflated, it constricts or even closes off the esophagus. One or more sensors located in the vicinity, detect at least one of the passage of food down the esophagus, or the acidity of the content of the esophagus or stomach, or the orientation of the subject, and adjust the balloon inflation in order to prevent reflux of the stomach content.

There is thus provided in accordance with a preferred embodiment of the present invention, a system for limiting the expansion of a hollow organ of a subject, comprising an inflatable balloon which, when inflated, covers an area at least the size of a significant part of a wall of the hollow organ, and which is adapted to be positioned proximate the wall, such that it limits expansion of the wall in the direction of the balloon. The hollow organ may preferably be the stomach of the subject, in which case the wall is a gastric wall, or it may be any one of the esophagus, the bladder, and a part of the lower GI tract.

If the hollow organ is the stomach, the balloon may preferably have a discoid shape with dimensions ranging from 10 cm. radius by 2 cm. thick to 30 cm. radius by 7 cm. thick, or it may have an ellipsoidal shape having a long axis ranging from 10 to 30 cm, a short axis ranging from about 5 to 10 cm and a thickness of between 2 and 10 cm. Alternatively and preferably, if the hollow organ is the esophagus, the balloon may preferably have a size of between 2 cm. and 4 cm. Furthermore, if the hollow organ is the stomach, the balloon may be preferably shaped to be positioned posterior to the stomach or in the Morrison pouch of the subject.

In accordance with yet another preferred embodiment of the present invention, the above described systems according may also comprise a flexible lumen for inflating the balloon, and an inflation port in fluid communication with the balloon through the flexible lumen, such that the balloon is inflatable from the inflation port.

In accordance with yet another preferred embodiment of the present invention, any of the above described systems may also preferably comprise an inflation pump in fluid connection to the balloon, such that the balloon is inflated by operation of the pump. The system may then also preferably comprise a sensor monitoring a physiological parameter related to the organ, and wherein an output signal derived from the sensor is utilized to operate the pump.

In accordance with still another preferred embodiment of the present invention, the pressure within the inflatable balloon of the above described systems may be utilized to determine the extension of organ

Furthermore, any of the above described systems may also comprise at least a second inflatable balloon adapted to be positioned adjacent a second wall of the hollow organ.

Additionally, in any of the above described systems, the balloon is preferably shaped so as to match a predetermined anatomical space of the subject into which it is intended to be inserted. Such a predetermined anatomical space is preferably selected so that movement of the balloon from the space is restricted.

In accordance with further preferred embodiments of the present invention, the balloon position proximate the wall of the hollow organ is extra-peritoneal. Furthermore, the balloon is preferably shaped and dimensioned to apply uniform pressure on tissues in its vicinity, such as to reduce the possibility of ischemic injury to the tissues.

There is further provided in accordance with still another preferred embodiment of the present invention, a system as described above, and in which the hollow organ is the esophagus, and wherein the balloon is adapted to control reflux through the esophagus. Such a system preferably also comprises at least one sensor detecting passage of food boluses through the esophagus. Additionally, such a system may also comprise at least one sensor detecting the pose of the subject, such that the balloon is inflated according to the pose of the subject.

There is even further provided in accordance with another preferred embodiment of the present invention a kit for the implantation of a system for the control of the expansion of a hollow organ of a subject, the kit comprising:

(i) at least one inflatable balloon for disposing adjacent the hollow organ,
(ii) a delivery tube for insertion into the subject to a region adjacent the hollow organ, the delivery tube being such that the inflatable balloon can be passed therethrough when in uninflated state, and
(iii) an inflation tube attached to the proximal end of the balloon, adapted for inflating the balloon after passage through the delivery tube.

In such a kit, the delivery tube is preferably adapted to be inserted into an extra-peritoneal region.

In accordance with yet another preferred embodiment of the present invention, there is provided a system for the control of the expansion of a hollow organ of a subject, the system comprising:

(i) at least one inflatable balloon for disposing adjacent the hollow organ,
(ii) at least one sensor outputting a signal relating to a physiological effect connected to the hollow organ, and
(iii) a pump utilizing the output to control the level of inflation of the at least one balloon.

The sensor may preferably be any one of an electrical activity sensor, a pressure sensor, a motion sensor, a displacement sensor and an ultrasonic sensor. It may preferably be located on or in proximity to the hollow organ. Additionally, the pump may preferably be either a peristaltic pump or a piston pump.

In accordance with still another preferred embodiment of the present invention, in any of the above described control systems, and where the hollow organ is a stomach, the sensor preferably outputs a signal relating to the degree of filling of the stomach. In such cases, the inflatable balloon itself may preferably be operative to detect the extension of the stomach by use of a pressure sensor to detect pressure changes in the balloon. Alternatively and preferably, the sensor may output a signal relating to the electrical activity of the stomach.

There is further provided in accordance with still another preferred embodiment of the present invention, a system for the control of gastric reflux in a subject, comprising:

(i) at least one inflatable balloon for disposing adjacent the esophagus of the subject,
(ii) at least one sensor determining when the subject is supine, and
(iii) a pump utilizing an output of the sensor to control the level of inflation of the at least one balloon.

Such a system may preferably further comprise a sensor for determining if the status of the gastric content is such that reflux thereof is likely. Additionally, the system may also comprise at least one sensor detecting passage of food boluses through the esophagus.

In accordance with a further preferred embodiment of the present invention, there is also provided a method of controlling the expansion of a hollow organ of a subject, the method comprising the steps of:

(i) providing at least one inflatable balloon for disposing adjacent the hollow organ,
(ii) inserting a delivery tube into the subject to a region adjacent the hollow organ, the delivery tube being such that the inflatable balloon can be passed therethrough when in uninflated state,
(iii) passing the inflatable balloon through the delivery tube to a position proximate the hollow organ, and
(iv) inflating the balloon after passage through the delivery tube, such that the expansion of the hollow organ is limited by the inflation of the balloon.

The region is preferably selected such that displacement of the balloon from the region is prevented. Furthermore, the method may also preferably comprise the steps of providing a flexible lumen for inflating the balloon and implanting subcutaneously an inflation port in fluid communication with the balloon through the flexible lumen, such that the balloon is inflatable from the inflation port.

### BRIEF DESCRIPTION OF THE DRAWINGS

The present invention will be understood and appreciated more fully from the following detailed description, taken in conjunction with the drawings in which:

FIGS. 1A to 1C schematically illustrate a cross-section of the abdominal wall at the location of the implanting of the device in the vicinity of the stomach, showing parts used for insertion and deployment of the inflatable balloon device of the present invention;

FIG. 2 illustrates schematically a cross section of the upper abdomen of the subject, showing two alternative locations for insertion of the inflatable device, either into the anterior abdominal wall, to limit the expansion of the stomach anteriorly and caudally using the kit of FIGS. 1A to 1C, or posterior to the stomach, and preferably implanted through a flank approach;

FIG. 3 schematically illustrate a frontal views of the subject's upper abdomen, showing a preferred size and location of the inflated device;

FIG. 4 schematically illustrates a preferred embodiment of the inflatable balloon of the present invention connected to the inflation port;

FIG. 5 illustrates schematically, according to a further preferred embodiment of the present invention, the balloon or balloons of the present invention connected to an implanted pump, controlled by a sensor, for regulating the inflated volume according to the sensor output; and

FIG. 6 illustrates schematically a cross section of the lower thorax of the subject, illustrating a further preferred embodiment of the present invention, in which one or more inflatable balloon devices are used for esophageal control, preferably as part of an automatic reflux suppression system.

### DETAILED DESCRIPTION OF PREFERRED EMBODIMENTS

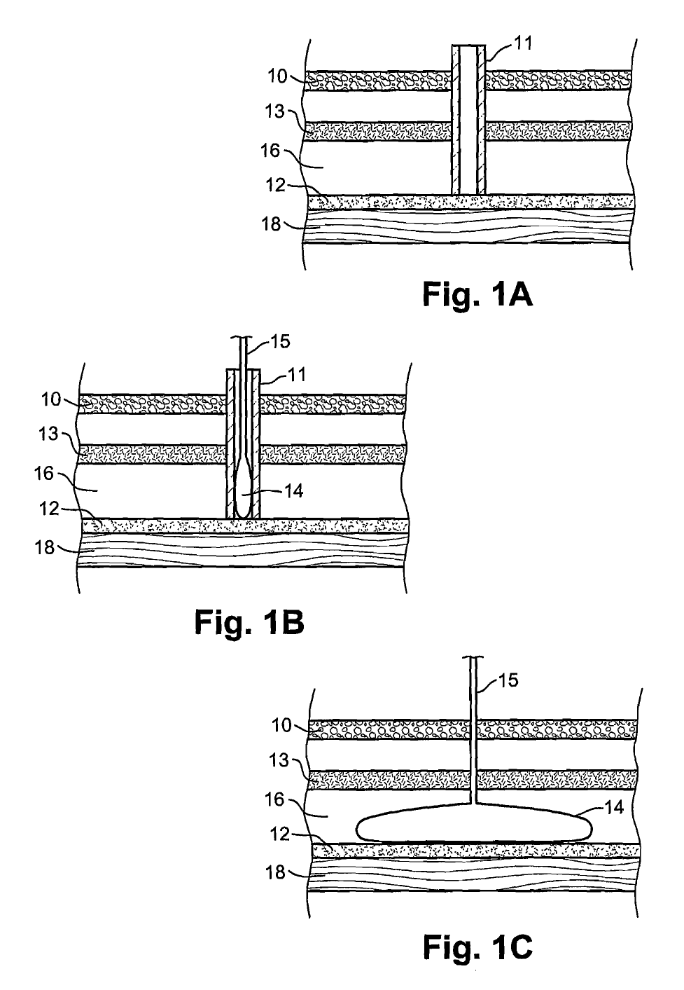

Reference is now made to FIGS. 1A to 1C, which are schematic illustrations of the parts used for insertion and deployment of the inflatable balloon device of the present invention, according to a first preferred embodiment, in which the device is disposed anterially to the stomach wall 18 and outside the peritoneum 12. The device is preferably introduced under ultrasound (US) or computerized tomography (CT) guidance, but other imaging modalities may be used such as: fluoroscopy, MRI, Scintigraphy, SPECT, PET, laparoscopy, trans-illumination, direct view or any combination thereof.

FIGS. 1A to 1C schematically illustrate a cross-section of the abdominal wall at the location of the implanting of the device in the vicinity of the stomach. The implantation is preferably executed by initially performing local anesthesia of the subcutaneous tissue fascia muscles 13, using a thin needle pro-peritoneally, preferentially under US or CT control. The pro-peritoneal space 16 may be developed by hydro-dissection, to create the intended anatomical space for the balloon, using a physiological solution. Under US or CT guidance, a guide wire is then preferably introduced through the needle into the created space. Then, a small incision is performed in the skin 10 at that location and a dilator is used to dilate the tract over the guide wire up to the properitoneal space, but preferably not penetrating the peritoneum 12. Then the dilator core is removed, and its sheath left in place. FIG. 1A schematically illustrates this stage of the implantation process, showing the site for the insertion of the inflatable balloon device and the dilator sheath 11. The inflatable balloon device 14 attached to a catheter 15, together with its enveloping sheath, are introduced into the pro-peritoneal space through the dilator sheath, as shown in FIG. 1B. The sheath is removed and the balloon is partially inflated to ascertain its proper position pro-peritoneally. This procedure is performed under continuous US, or CT guidance in order to ascertain that the balloon 14 is inflated pro-peritoneally and situated in front of the anterior wall 18 of the stomach, until it is inflated to its desired size, as shown in FIG. 1C. A contrast medium such as a hyper-echoic material in the case of US, or a radio-opaque material in the case of a CT, may be introduced into the balloon in order to improve visualization. Additionally, a contrast medium may be swallowed by the patient in order to better delineate the stomach, and to increase the stomach volume similarly to that achieved during a meal, in order to determine the dynamic relation of the balloon to the stomach and the degree of limitation of gastric filling, or to determine the volume at which a sensation of satiety and/or nausea occurs.

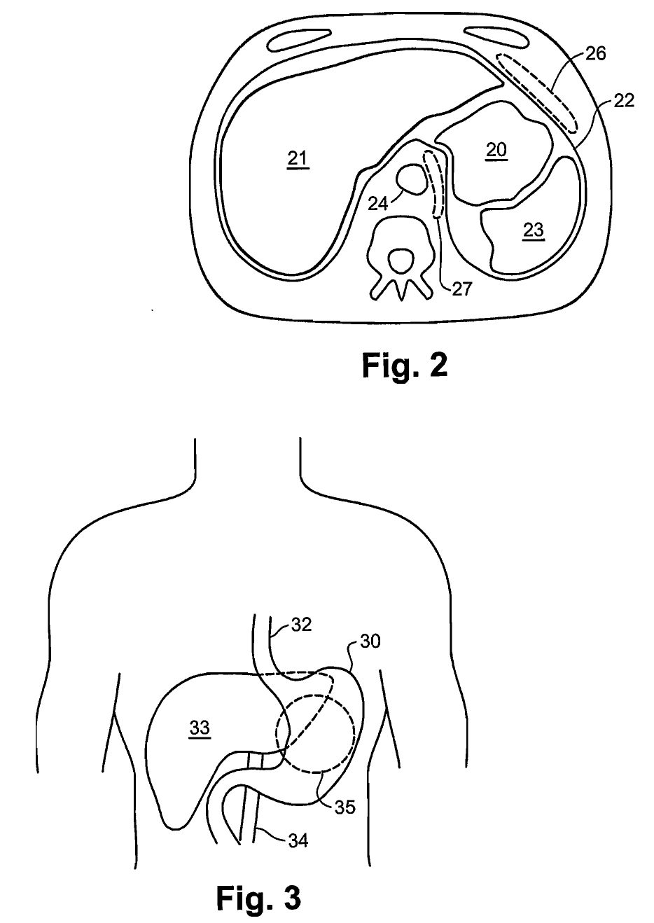

Reference is now made to FIG. 2, which is a schematic cross section of the upper abdomen of a subject, showing the stomach 20, the liver 21, the peritoneum 22, the spleen 23 and the aorta 24. The position of the organs and other anatomical parts is merely schematic in order to show a gastric embodiment of the present invention and its implementation, and is not meant to be an accurate representation of the subject's anatomy. FIG. 2 illustrates schematically two alternative locations for insertion of the inflatable device of the present invention adjacent to the stomach 20 to limit its expansion. The first location 26 is in the anterior abdominal wall, to limit the expansion of the stomach anteriorly and caudally. Insertion is performed using the kit of FIGS. 1A to 1C. The device is introduced into a space within the upper abdomen above the umbilicus between the peritoneum and the anterior abdominal muscles, or between the posterior rectus muscle sheath and rectus muscle. Since the location is pro-peritoneal, the balloon, when implanted, cannot readily change its position. Location of the inflatable device outside of the peritoneum also avoids potential complications engendered by surgical penetration of the peritoneum.

According to a further preferred embodiment of the present invention, the inflatable device as described above, can be disposed posterior to the stomach, as shown in the alternative location 27 of FIG. 2, and is preferably implanted through a flank approach. The preferred route is from the left side flank. The patient is positioned on his right side with the left side up. A needle is inserted, preferably under CT guidance, through the left flank, laterally to the para-vertebral muscles, passing posterior or anterior to the upper pole of the left kidney and reaching above the pancreas body. In cases where there is not a straight path to this location, hydro-dissection may be used to separate the tissues. Alternatively and preferably, a deflectable catheter and/or deflectable guide-wire may be used to reach this location. After dilation of the tract by a specially designed dilator, a balloon catheter is introduced over the guide wire to the proper location above the pancreas body, or in front of the pancreas and behind the stomach. This position may be posterior to the Morrison's pouch or within Morrison's pouch.

Reference is now made to FIG. 3 which schematically illustrates frontal views of the subject's upper abdomen, showing the stomach 30, the liver 31 the esophagus 32 and the portal vein 33. A preferred shape of the balloon 35 when fully inflated is shown. When inflated, the balloon preferably covers an area equivalent to a significant part of the stomach wall, and may preferably be discoid in shape, ranging in sizes from a radius of approximately 10 cm and a thickness of approximately 2 cm. for the smallest sized patients, to a size of 30 cm radius by 7 cm thick for the largest sized patients. Alternatively and preferably, the inflated balloon may be of ellipsoidal shape, with a long axis of the order of 10 to 30 cm, a short axis of between about 5 and 10 cm and a thickness of between 2 and 10 cm.

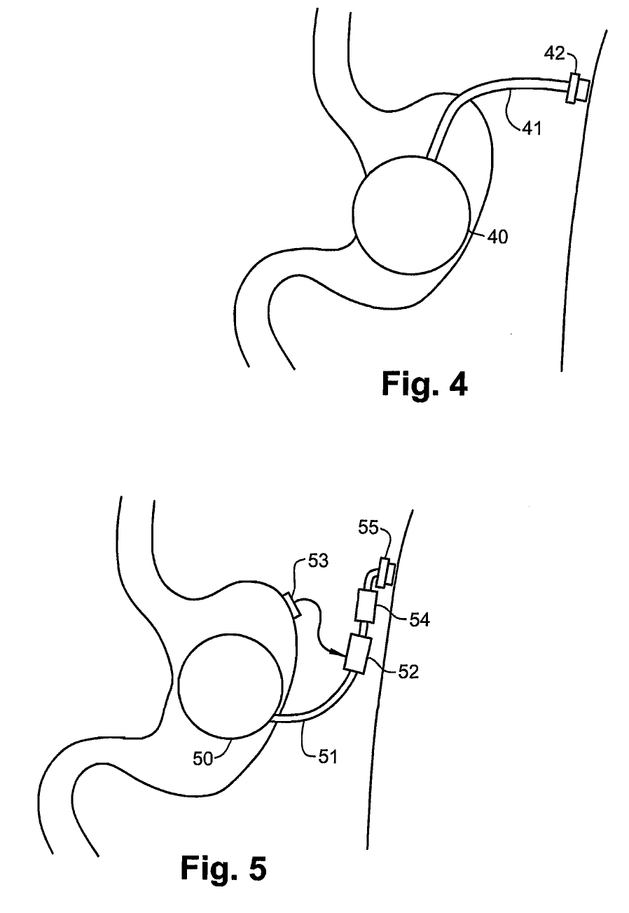

Reference is now made to FIG. 4, which is a schematic illustration according to a preferred embodiment of the present invention, of a gastric control balloon 40 connected by means of a catheter 41 to is filled to an inflation port 42 so that the balloon can be filled to its predetermined volume, or its volume adjusted later according to the needs of the subject. The inflation port is preferably situated subcutaneously, such that the inlet valve is easily accessible to the physician for injecting the inflating fluid. The inflation port is preferably located in an easily accessible location such as the upper abdomen for an anterior placed balloon, or in the subcutaneous tissue of the flank for a posterior placed balloon. The inflation port 42 preferably comprises a rigid frame made of a biocompatible metal such as stainless steel, nitinol or titanium, or a plastic material, covered by a self sealing skin or membrane such as silicone membrane. The port may preferably be releasably connected to the catheter of the device by a connector, not shown in FIG. 4. After introduction of the inflatable device in its proper position, the device catheter 41 is connected to the port. The balloon is partially inflated with an inflation fluid such as saline, which may also contain a contrast material. The site and size of the balloon are ascertained by an imaging modality such as US or CT.

Using the percutaneous inflation port, the balloon can be easily inflated in progressive steps during successive sessions, as needed to obtain the optimal limitation of expansion of the stomach.

Although the above embodiments have been described in terms of a single balloon, it is to be understood that the invention is also meant to cover the use of more than one balloon, which may all be connected to the same inflation port, or each of which may have its own inflation port. Alternatively and preferably, one or more balloons may be inserted within the anterior abdominal wall and one or more balloons may be inserted within the posterior abdominal wall.

Reference is now made to FIG. 5, which illustrates schematically, according to a further preferred embodiment of the present invention, the balloon 50 or balloons connected by means of a filling tube or catheter 51 to an implanted pump 52 that can regulate the inflated volume as desired. The pump is preferably controlled by means of a controller, which receives its command inputs from a sensor 53, which transmits gastric-related data to the controller. The controller may be a stand alone unit, or it may preferably be incorporated into the pump unit or into the sensor unit, and for this reason is not shown as a separate unit in FIG. 5. The sensor preferably provides data concerning the degree of filling of the stomach, such as by sensing the change in pressure in the balloon, or by sensing the electrical activity of the stomach using electrodes attached to the implanted balloon, or by means of electrodes otherwise implanted into the anterior or posterior abdominal wall near the stomach wall. Alternatively and preferably, other sensing modalities may be used, such as motion sensors, pressure sensors, deformation sensors, displacement sensors, position sensors based on the piezoelectric effect, on infrared or radiofrequency, or ultrasonic sensors, such as are known in the art. Such sensors may be implanted in the anterior or posterior abdominal wall near the stomach wall. The pump 52 used may preferably be a peristaltic pump, a piston pump, or any other small pump that is preferably implanted within the subcutaneous tissue of the abdominal wall. The fluid for topping up the balloon is preferably contained in a reservoir 54, preferably also implanted subcutaneous, into which excess fluid is also returned when the control system sees the need to reduce the balloon volume. The energy source, such as a battery, should also preferably be implanted within the subcutaneous tissue and connected to the pump system and sensing unit. A subcutaneous inflation port 55 is also provided for the initial filling, and for maintenance filling when required.

When necessary, the balloon, balloons, catheter and inflation ports can be removed under local anesthesia by palpating the inflation port, making an incision above it, removing it and then removing the connected catheter and balloon after deflation. Use of a material such as medical grade silicone—inducing minimal fibrosis—makes removal easy.

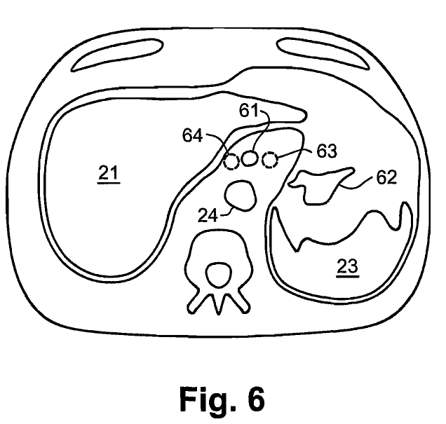

Reference is now made to FIG. 6, which illustrates schematically a further preferred embodiment of the present invention, in which one or more inflatable balloon devices 63, 64, may be inserted near and laterally to the esophagus. FIG. 6 illustrates schematically a cross section of the abdomen of the subject, taken at a higher plane than that of FIG. 2, such that the esophagus 61 is shown, and the tip of the stomach 62. One or more inflatable balloon devices 63, 64 are used for esophageal control as part of a reflux suppression system. Such balloons are smaller than those used for gastric control, since the organ to be controlled is that much smaller, and typical sizes are from 2 to 4 cm. The balloons may also preferably be spherical. These esophageal devices may be inserted percutaneously, para-vertebrally, into the posterior mediastinum, through the anterior thoracic wall, or into the neck, under imaging guidance. This imaging may be CT, MRI, US, fluoroscopy, scintigraphy, SPECT, PET, laparoscopy, mediastinoscopy, trans-illumination, direct view or any combination thereof. Alternatively, these devices may be introduced paravertebrally, for example, below the 12th. rib, by the same guidance means.

The balloon itself, or one of the balloons if there is more than one, may serve for sensing the peristaltic activity of the esophagus and passage of food boluses, since the pressure within the balloon itself can provide an indication of the outward motion of the esophagus with the passage of food. This sensing is utilized for detecting the quantity of food ingested and or its constituents. Alternatively and preferably, dedicated sensors may be inserted, such as electrodes sensing electrical activity, pressure sensors, movement sensors, displacement sensors, ultrasonic sensors, or any other such sensor known in the art. The device may preferably include connection to an inflation port, with any of the control features described above in relation to the gastric devices, such as sensors, controllers, pumps and fluid reservoirs.

Such esophageal balloons may also serve for preventing reflux of the gastric content into the esophagus, which is a cause of a disease known as gastroesophageal reflux disease (GERD). Preferably under local anesthesia, the balloons are inflated progressively percutaneously to the desired volume, preferably through a percutaneously situated inflation port. The desired volume may preferably be determined as that at which the pressure exerted by the balloon on the esophagus is not high enough to impede the swallowing of food boluses, yet is sufficient to prevent the reflux, which generally occurs with only slight intra-gastric overpressure, or even just under the effects of gravity when the subject is supine. A suitable contrast substance may be added to the balloon or balloons to better delineate them in the imaging means. The level of reflux, and the success in limiting it as a function of the balloon inflation, may be evaluated by giving to the patient a meal containing a contrast material or a radio-labeled meal and determining the reflux of the meal to the esophagus when the patient is supine, or under the effect of increased intra-abdominal pressure by straining. The evaluation can preferably be performed by suitable imaging means such as fluoroscopy, CT, MRI, scintigraphy or SPECT.

Alternatively and preferably, the pH of the lower esophagus may be monitored by the use of dedicated pH probes and the presence and degree of reflux ascertained and controlled by control of the balloon inflation.

In accordance with a further preferred embodiment of the present invention, an automatic reflux suppression system is provided, in which the esophageal balloon or balloons are connected to an implanted pump, similar to that shown in the gastric embodiment of FIG. 5, that controls the inflating volume introduced into the balloon or balloons, according to an input provided by a reflux sensor. According to a further preferred embodiment of the present invention, one or more positional sensors may be implanted in the subject, and can determine when the patient is supine. Alternatively, other sensors such as ultrasonic or displacement sensors may be used. This is desirable since GERD occurs mainly when lying down. In this case, the pump is activated to increase the volume in the balloon or balloons near the esophagus, especially when the subject is supine, thus preventing reflux of gastric content into the esophagus. The pump used may be a peristaltic pump, a piston pump, or any other small pump that may be implanted within the subcutaneous tissue of the abdominal wall. An energy source should be implanted also within the subcutaneous tissue and connected to the pump system and sensing unit. These additional pumping and control components are essentially similar functionally to those shown in FIG. 5 for the gastric case, except that the components are selected for this reflux control embodiment, such that the balloon or balloons are correspondingly smaller and esophageally positioned, and the sensor is preferably a position sensor, which can be located anywhere on the upper body of the subject. Alternatively and preferably, the balloon or balloons can be controlled by inputs both from a positional detector, and from a gastric content sensor, since every act of reclining need not be accompanied by reflex suppression if the gastric conditions do not indicate that such reflux is likely.

Although the inflatable balloon devices of the present invention have been described for use in applications related to gastric control, it is to be understood that the invention is not limited thereto, but is also meant to include their use for implantation near other hollow organs such as the urinary bladder, for treating urinary retention or incontinence, the colon, for treating constipation, the anal canal for treating fecal incontinence, and other suitable locations.

It is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

1. A system for the control of the expansion of a hollow organ of a subject, comprising an inflatable balloon which, when inflated, covers an area at least the size of a significant part of a wall of said hollow organ, and which is adapted to be positioned proximate said wall, such that it limits expansion of said wall in the direction of said balloon.

2. A system according to claim 1 and wherein said hollow organ is the stomach of the subject, and wherein said wall is a gastric wall.

3. A system according to claim 1 and wherein said hollow organ is any one of the esophagus, the bladder, and a part of the lower GI tract.

4. A system according to claim 1 and also comprising:
a flexible lumen for inflating said balloon; and
an inflation port in fluid communication with said balloon through said flexible lumen, such that said balloon is inflatable from said inflation port.

5. A system according to claim 2 and wherein said balloon has a discoid shape having dimensions ranging from 10 cm. radius by 2 cm. thick to 30 cm. radius by 7 cm. thick.

6. A system according to claim 2 and wherein said balloon has an ellipsoidal shape having a long axis ranging from 10 to 30 cm, a short axis ranging from about 5 to 10 cm and a thickness of between 2 and 10 cm.

7. A system according to claim 3 and wherein said hollow organ is said esophagus, and wherein said balloon has a size of between 2 cm. and 4 cm.

8. A system according to claim 2 and wherein said balloon is shaped to be positioned posterior to the stomach or in the Morrison pouch of the subject.

9. A system according to claim 1 and also comprising an inflation pump in fluid connection to said balloon, such that said balloon is inflated by operation of the pump.

10. A system according to claim 9 and also comprising a sensor monitoring a physiological parameter related to said organ, and wherein an output signal derived from said sensor is utilized to operate said pump.

11. A system according to claim 1 and wherein the pressure within said inflatable balloon is utilized to determine the extension of said organ

12. A system according to claim 1 and also comprising at least a second inflatable balloon adapted to be positioned adjacent a second wall of said hollow organ.

13. A system according to claim 1 and wherein said balloon is shaped so as to match a predetermined anatomical space of the subject into which it is intended to be inserted.

14. A system according to claim 13 and wherein said predetermined anatomical space is such that movement of said balloon from said space is restricted.

15. A system according to claim 1 and wherein said balloon position proximate said wall is extra-peritoneal.

16. A system according to claim 1 and wherein said balloon is shaped and dimensioned to apply uniform pressure on tissues in its vicinity, such as to reduce the possibility of ischemic injury to said tissues.

17. A system according to according to claim 1 and wherein said hollow organ is the esophagus of the subject, and wherein said balloon is adapted to control reflux through said esophagus.

18. A system according to claim 17 and also comprising at least one sensor detecting passage of food boluses through said esophagus.

19. A system according to claim 17 and also comprising at least one sensor detecting the pose of the subject, such that said balloon is inflated according to the pose of the subject.

20. A kit for the implantation of a system for the control of the expansion of a hollow organ of a subject, the kit comprising:
at least one inflatable balloon for disposing adjacent said hollow organ;
a delivery tube for insertion into the subject to a region adjacent said hollow organ, said delivery tube being such that said inflatable balloon can be passed therethrough when in uninflated state; and
an inflation tube attached to the proximal end of said balloon, adapted for inflating said balloon after passage through said delivery tube.

21. The kit according to claim 20, and wherein said delivery tube is adapted to be inserted into an extra-peritoneal region.

22. A system for the control of the expansion of a hollow organ of a subject, said system comprising:
at least one inflatable balloon for disposing adjacent said hollow organ;
at least one sensor outputting a signal relating to a physiological effect connected to said hollow organ; and
a pump utilizing said output to control the level of inflation of said at least one balloon.

23. A system according to claim 22, and wherein said sensor is any one of an electrical activity sensor, a pressure sensor, a motion sensor, a displacement sensor and an ultrasonic sensor.

24. A system according to claim 22, and wherein said sensor is located on or in proximity to said hollow organ.

25. A system according to claim 22, and wherein said pump is either one of a peristaltic pump and a piston pump.

26. A system according to claim 22, and wherein said hollow organ is a stomach, and said sensor outputs a signal relating to the degree of filling of the stomach.

27. A system according to claim 22, and wherein said inflatable balloon is operative to detect the extension of said stomach by use of a pressure sensor to detect pressure changes in said balloon.

28. A system according to claim 22, and wherein said hollow organ is a stomach, and said sensor outputs a signal relating to the electrical activity of the stomach.

29. A system for the control of gastric reflux in a subject, comprising:
at least one inflatable balloon for disposing adjacent the esophagus of the subject;
at least one sensor determining when the subject is supine; and
a pump utilizing an output of said sensor to control the level of inflation of said at least one balloon.

30. A system according to claim 29 and further comprising a sensor for determining if the status of the gastric content is such that reflux thereof is likely.

31. A system according to claim 29 and further comprising at least one sensor detecting passage of food boluses through said esophagus.

32. A method of controlling the expansion of a hollow organ of a subject, said method comprising the steps of:
providing at least one inflatable balloon for disposing adjacent said hollow organ;
inserting a delivery tube into the subject to a region adjacent said hollow organ, said delivery tube being such that said inflatable balloon can be passed therethrough when in uninflated state;
passing said inflatable balloon through said delivery tube to a position proximate said hollow organ; and
inflating said balloon after passage through said delivery tube, such that the expansion of said hollow organ is limited by the inflation of said balloon.

33. A method according to claim 32 and wherein said region is selected such that displacement of said balloon from said region is prevented.

34. A method according to claim 32 and also comprising the steps of providing a flexible lumen for inflating said balloon and implanting subcutaneously an inflation port in fluid communication with said balloon through said flexible lumen, such that said balloon is inflatable from said inflation port.
